(19) United States
(12) Patent Application Publication
(10) Pub. No.: US 2007/0059346 A1
(43) Pub. Date: Mar. 15, 2007

(54) Film comprising therapeutic agents

(76) Inventor: Todd Maibach, Menlo Park, CA (US)

Correspondence Address: Kirkpatrick & Lockhart Preston Gates Ellis LLP, 1735 NEW YORK AVENUE, NW, SUITE 500, WASHINGTON, DC, 20006, US

(21) Appl. No.: 10/562,633

(22) PCT Filed: Jun. 30, 2004
(86) PCT No.: PCT/US04/21038

§ 371 Date: Jun. 9, 2006

Related U.S. Application Data
(60) Provisional application No. 60/484,009, filed on Jul. 1, 2003.
(60) Provisional application No. 60/497,426, filed on Aug. 21, 2003.

(51) Int. Cl. A61K 9/70; A61K 31/21; A61K 31/60; A61K 31/192; A61K 31/405; A61K 31/16

(52) U.S. Cl. 424/443; 514/509; 514/410; 514/165; 514/569; 514/570; 514/629

(57) ABSTRACT

The present invention is related to the composition and methods of manufacture of orally-dissolvable, edible films as a vehicle for the non-invasive administration of nitroglycerin, as well as other therapeutic agents either with or without nitroglycerin, through the mucosal tissues of the oral cavity. The films include a water soluble film-forming polymer such as pullulan. Methods for producing the films are also disclosed.

### CROSS REFERENCE TO RELATED APPLICATION

The present application claims the benefit, under 35 U.S.C. § 119, of U.S. Provisional Patent Application Ser. No. 60/484,009, filed 1 Jul. 2003, and U.S. Provisional Patent Application Ser. No. 60/497,426, filed 21 Aug. 2003, the contents of which are incorporated herein by reference.

### FIELD OF THE INVENTION

This invention relates to the administration of therapeutic agents including nitroglycerin, via consumable, edible films.

### BACKGROUND OF THE INVENTION

Nitroglycerin is a powerful vasodilator used to prevent chest pain (angina pectoris) by relaxing the smooth muscle of blood vessels in the heart, increasing blood flow and oxygen to the heart muscle, and reducing the pumping force the heart must exert to circulate blood through the body. This reduction in the heart's workload relieves the pain of angina pectoris. Nitroglycerin also finds additional utility in controlling blood pressure in perioperative hypertension, or hypertension resulting from intratracheal intubation, anesthesia, skin incision, sternotomy, cardiac bypass, and postsurgical recovery, in addition to producing controlled hypotension during surgery.

Existing methods of administration of nitroglycerin include a nitroglycerin pump-spray, nitroglycerin sublingual tablet, nitroglycerin sustained released tablets, nitroglycerin transdermal patches, nitroglycerin 2% ointment, and an intravenous nitroglycerin drip. However, each of these methods have inherent drawbacks.

Oral administration is probably the most prevalent method of administering nitroglycerin because of its convenience. It is generally non-threatening, painless, and simple to accomplish for most patients. Nevertheless, the oral administration of nitroglycerin suffers from several disadvantages. Specific problems associated with the oral administration of compressed sustained-release nitroglycerin tablets include friability, content uniformity, such as weight and dosage variations, migration of nitroglycerin to other tablets, the storage container and container components and the resulting potency loss.

A further problem with oral administration in pill form is that the rate of absorption of the drug into the bloodstream after swallowing varies from patient to patient. The absorption of the drug is dependent upon the movement of the drug from the stomach to the small and large intestines and the effects of secretions from these organs and on the resulting pH within the stomach and intestines. Anxiety and stress can dramatically reduce these movements and secretions, prevent or reduce the final effects of the drug, and delay onset of the drug's effects. Most significant is the fact that there is normally a substantial delay between the time of oral administration and the time that the therapeutic effect of the drug begins.

An additional disadvantage of oral pill form administration is that many drugs almost immediately experience metabolism or inactivation. The veins from the stomach and the small and large intestines pass directly through the liver. Thus, drugs entering the bloodstream must first pass through the liver before distribution into the general blood circulation. More than sixty percent of most drugs (and essentially one hundred percent of certain drugs) are removed from the patient's bloodstream during this “first pass” through the liver. The result is that oral pill form administration is impractical for many drugs, particularly cardiovascular-acting drugs that are used for rapid onset in critical care situations.

In order to avoid some of the disadvantages of oral administration, injection is frequently used. Injecting nitroglycerin intravenously results in rapid entry of the drug into the patient's bloodstream. In addition, this type of delivery avoids the removal of large quantities of the drug by the patient's liver. As a result, less total drug is usually needed compared to orally distributed to various portions of the patient's body before exposure to the liver. However, most patients, particularly children and geriatric adults, have an aversion to injections. In some patients, this aversion may be so pronounced as to make the use of injections a serious concern. Since intense psychological stress can exacerbate a patient's debilitated condition, it sometimes becomes undesirable to use injections where the patient is seriously ill or suffers from a debilitating condition or injury.

Another method of administration of therapeutic agents, such as nitroglycerin, includes the transdermal patch. In this method of administration, a dose of nitroglycerin is administered by absorption through the dermal layers into the blood stream. However, a serious disadvantage of the transdermal patch method of nitroglycerin administration is the development of a drug tolerance within a twenty-four (24) hour period when patches are worn continuously, subsequently reducing the effectiveness of the medication. Revised labeling approved by FDA recommended a dosing schedule alternating a daily patch-on period of 12 to 14 hours a day with a patch-off period of 10 to 12 hours, making this time consuming and easily forgotten. Moreover, the patch cannot be used on parts of the body with hair, cuts, abrasions, calluses or scars, and may lead to skin irritation where the patch is applied.

Some investigators have suggested that it may be possible to administer medication through the buccal mucosa of the cheek pouch or by sublingual administration. See, U.S. Pat. No. 4,671,953, the entire content of which is incorporated by reference herein. Such administration through the mucosal tissues of the mouth, pharynx, and esophagus of therapeutic drugs possesses a distinct usefulness. Administration of drugs by this route does not expose the drug to the gastric and intestinal digestive juices. In addition, the drugs largely bypass the liver on the first pass through the body, thereby avoiding additional metabolism and/or inactivation of the drug.

Generally the drugs which are administered by any of the methods described above have an unpleasant taste. As a result, in order to allow for buccal or sublingual administration through the oral mucosal tissues, it is also necessary to incorporate the drug into some type of pleasant tasting mass, such as a “candy” matrix.

For effective application of the drug, a candy product may contain the drug uniformly distributed throughout in order to ensure uniform levels of medication. Alternatively, for some applications, varying concentrations within known and controlled ranges may be desired to vary the rate of drug administration. Difficulties are encountered in attempting to blend solid drugs in a uniform or otherwise carefully controlled manner. Many drugs are insoluble, or only partially soluble, in one or more of the ingredients of the hard candy base. Thus, the resultant product is often found to be lacking in uniform or controlled distribution of the drug. Moreover, sublingual tablets also experience issues related to inter-tablet migration of nitroglycerin, similar to the sustained-release tablet methodology, which can produce a high degree of weight and dose variation between tablets.

Furthermore, many presently available medicated candy lozenges tend to crumble when placed in the mouth. As a result, uniform release of the drug into the mucosal tissues does not take place. Rather, the crumbled lozenge is mostly chewed, and swallowed, and the drug enters the bloodstream through the stomach and intestines as described above. Thus, it will be appreciated that candy lozenges have very definite limitations for use in the administration of a drug through the oral mucosal tissues. As a result, lozenges have not been used to administer potent, fast-acting drugs, such as drugs that affect the central nervous system, the cardiovascular system, or the renal vascular system.

While the administration of certain drugs through the oral mucosal tissues has shown promise, development of a fully acceptable method for producing a medication in a desirable form and administering the medication has been elusive.

It would be an important advancement in the art of orally administering potent, fast-acting drugs, if suitable methods and compositions provided a precise dosage to a precise effect in every patient. It would be a further advancement in the art to provide methods and compositions for uniformly incorporating drugs (including insoluble drugs) into a soluble matrix without heating the mixture to the point that degradation occurs.

A need, therefore, exists for an improved vehicle for the administration of therapeutic agents, such as nitroglycerin, beyond existing preparations.

### SUMMARY OF THE INVENTION

The invention provides a physiologically acceptable edible or consumable film, which is particularly well adapted to rapidly dissolve in the mouth of a patient. In an embodiment of the present invention, the film comprises nitroglycerin. In another embodiment, the film comprises nitroglycerin and at least one additional pharmaceutically active agent.

In another embodiment of the present invention, the edible or consumable film comprises a therapeutic agent or combination of two or more therapeutic agents, wherein the therapeutic agents include, but are not limited to agents useful as anti-microbial agents, non-steroidal anti-inflammatory drugs, anti-inflammatory drugs, anti-tussives, decongestants, anti-histamines, expectorants, anti-diarrheals, H₂-antagonists, proton pump inhibitors, general nonselective CNS depressants, general nonselective CNS stimulants, drugs that selectively modify CNS function, anti-parkinsonism drugs, narcotic-analgesics, analgesic-antipyretics, psychopharmacological drugs, anti-hypertension and cardiovascular treatment agents, dermatological agents, glucocorticoids and steroids, antimalarial and anti-parasitic agents, anti-fungal agents, anti-periodontitis agents, emetic agents, treatments for gout, treatments for glaucoma, treatments for attention-deficit hyperactivity disorder, pre-treatment and treatment for exposure to chemical weapons, treatments for acute radiation exposure, narcotic analgesic agents, hemostatic agents, treatments for Sjörgren's Syndrome and smoking cessation agents.

The invention is also directed to a method for producing a supple, non-self-adhering film especially suitable for oral delivery of nitroglycerin. The method comprises mixing at least one film forming agent with an aqueous solution to provide a hydrated polymer gel; casting the hydrated polymer gel on a substrate; and allowing the cast gel to solidify to provide a film. In another embodiment, the nitroglycerin or other therapeutic agent or agents are added to one or more of the components of the mixture prior to forming the hydrated polymer gel.

### DETAILED DESCRIPTION OF THE INVENTION

It is understood that the present invention is not limited to the particular methodology, protocols, reagents, etc. described herein, as these may vary. It is also to be understood that the terminology used herein is used for the purpose of describing particular embodiments only, and is not intended to limit the scope of the present invention. It must be noted that as used herein and in the appended embodiments, the singular forms “a,” “an,” and “the” include plural reference unless the context clearly dictates otherwise.

Unless defined otherwise, all technical and scientific terms used herein have the same meanings as commonly understood by one of ordinary skill in the art to which this invention belongs. Preferred methods, system components, and materials are described, although any methods and materials similar or equivalent to those described herein can be used in the practice or testing of the present invention. All references cited herein are incorporated by reference herein in their entirety.

All publications and patents mentioned herein are incorporated herein by reference for the purpose of describing and disclosing, for example, the system components and methods that are described in the publications, which might be used in connection with the presently described invention. The publications discussed herein are provided solely for their disclosure prior to the filing date of the present application. Nothing herein is to be construed as an admission that the inventors are not entitled to antedate such disclosure by virtue of prior invention or for any other reason.

The present invention relates to the composition and methods of manufacture of orally-dissolvable, edible or consumable films as a vehicle for the non-invasive administration of nitroglycerin through the mucosal tissues of the oral cavity including, but not limited to, the mouth, pharynx, and esophagus. The present invention also relates to the composition and methods of manufacture of orally-dissolvable, edible or consumable films as a vehicle for the non-invasive administration of a variety of therapeutic agents, which may or may not also include nitroglycerin in the film, through the mucosal tissues of the oral cavity including, but not limited to, the mouth, pharynx, and esophagus.

One embodiment of the present invention is a physiologically acceptable film that is particularly well adapted to dissolve in a mouth of a patient to deliver a nitroglycerin agent that can be used as an effective tool in the treatment or prevention of diseases or conditions including, but not limited to, angina pectoris, ventricular arrhythmia, supraventricular arrhythmia, and other cardiovascular conditions and diseases, or any other disease or condition that may be treated with nitroglycerin. This film may comprise any edible or consumable polymer or film forming agent and nitroglycerin.

In another embodiment of the present invention, the edible or consumable film comprises a therapeutic agent or combination of two or more therapeutic agents, wherein the therapeutic agents include, but are not limited to, agents useful as anti-microbial agents, non-steroidal anti-inflammatory drugs, anti-inflammatory drugs, anti-tussives, decongestants, anti-histamines, expectorants, anti-diarrheals, H₂-antagonists, proton pump inhibitors, general nonselective CNS depressants, general nonselective CNS stimulants, drugs that selectively modify CNS function, anti-parkinsonism drugs, narcotic-analgesics, analgesic-antipyretics, psychopharmacological drugs, anti-hypertension and cardiovascular treatment agents, dermatological agents, glucocorticoids and steroids, antimalarial and anti-parasitic agents, anti-fungal agents, anti-periodontitis agents, emetic agents, treatments for gout, treatments for glaucoma, treatments for attention-deficit hyperactivity disorder, pre-treatment and treatment for exposure to chemical weapons, treatments for acute radiation exposure, narcotic analgesic agents, hemostatic agents, treatments for Sjörgren's Syndrome and smoking cessation agents. These films may or may not also comprise nitroglycerin.

U.S. Pat. No. 5,518,902 to Ozaki et al. (Hayashibara), the entire contents of which are incorporated by reference herein, discloses high pullulan content products, such as edible films, dentifrices and pharmaceuticals (column 3, lines 44-56 and Example B-8). The products can include a variety of ingredients in addition to pullulan, such as other polysaccharides, polyhydric alcohols, antiseptics and flavor-imparting agents (column 4, line 58 to column 5, line 11).

U.S. Pat. No. 5,411,945 to Ozaki et al. (Hayashibara), the entire contents of which are incorporated by reference herein, discloses a pullulan binder and products produced therewith, including edible films (Example B-2). The products can include a variety of ingredients in addition to pullulan, such as other polysaccharides, antibacterial agents, flavor-imparting agents and pharmaceutically active substances (column 4, lines 5-15).

U.S. Pat. No. 4,851,394 to Kubodera, the entire contents of which are incorporated by reference herein, discloses glucomannan/polyhydric alcohol edible films, which can comprise pullulan (column 3, line 59 to column 4, line 21). The films are contrasted with existing pullulan-based films, which are said to lack resistance to water (column 1, lines 40-44).

U.S. Pat. No. 3,784,390 Hijiya et al., the entire contents of which are incorporated by reference herein, discloses pullulan films and their use in coating and packing materials for foods, pharmaceuticals and other oxygen sensitive materials. All of the examples in this patent teach mixing pullulan in hot water.

U.S. Pat. No. 4,623,394 Nakamura et al., the entire contents of which are incorporated by reference herein, discloses a gradually disintegrable molded article that can be a film made with pullulan. The articles contain a particular heteromannan, which can be locust bean gum.

U.S. Pat. No. 4,562,020 Hijiya et al., the entire contents of which are incorporated by reference herein, discloses a process for producing a self-supporting film of a glucan, which can be pullulan.

U.S. Pat. No. 5,569,482 to Naga et al., the entire contents of which are incorporated by reference herein, discloses a method for the manufacture of an edible proteinaceous film from various sources of soybean protein.

U.S. Pat. No. 5,288,497 to Stanley et al., the entire contents of which are incorporated by reference herein, discloses methods of manufacture for the production and administration of lipophilic and nonlipophilic drugs capable of absorption through the mucosal tissues of the mouth, pharynx, and esophagus.

U.S. Pat. No. 6,020,002 to Fuisz Technologies, Ltd., the entire contents of which are incorporated by reference herein, discloses a shearform matrix based composition which may be formed into tablets (column 4 though column 7, line 47). The matrix is formed using a flash-shear process disclosed therein. No mention is made of producing a film using the disclosed matrix, and the matrix disclosed requires heating.

U.S. Pat. No. 6,337,082 to Fuisz et al., the entire contents of which are incorporated by reference herein, discloses matrices which can be used to deliver therapeutic agents, and to make food items.

WO 03/011259, the entire contents of which are incorporated by reference herein, discloses maltodextrin edible films for release into the oral cavity.

WO 03/043659, the entire contents of which are incorporated by reference herein, discloses an edible film comprised of a hydrocolloid film-forming agent that rapidly disintegrates when placed in the mouth to release an active agent.

WO 02/43657, the entire contents of which are incorporated by reference herein, discloses pullulan-free edible film compositions and methods for making same.

WO 02/02645, the entire contents of which are incorporated by reference herein, discloses a process for using cold-water soluble P-glucan to create a gel for use in numerous applications, including the formation of an edible film.

WO 99/17753, the entire contents of which are incorporated by reference herein, discloses rapidly dissolving films for delivery of drugs to be adsorbed in the digestive tract.

WO 98/26780, the entire contents of which are incorporated by reference herein, discloses a flat, foil, paper or wafer type presentation for the application and release of active substances in the buccal cavity. The specific active ingredient disclosed in WO 98/26780 is buprenorphine.

WO 98/20862, the entire contents of which are incorporated by reference herein, discloses a film for use in the oral cavity that can contain a cosmetic or pharmaceutical active substance.

U.S. Application Serial No. 2003/0107149, the entire contents of which are incorporated by reference herein, discloses a method for making films to be used for oral drug delivery. No mention is made of delivering nitroglycerin.

WO 98/26763, the entire contents of which are incorporated by reference herein, discloses a flat, foil, paper or wafer like presentation for release of active substances into the buccal cavity. The particular active substance disclosed is apomorphine.

U.S. Patent Appl. Serial No. 2003/00080008, the entire contents of which are incorporated by reference herein, discloses a consumable film with high concentrations of anti-microbial agents and essential oils.

U.S. Patent Appl. Serial No. 2003/0035841, the entire of contents of which are incorporate by reference herein, discloses an edible film for use in the oral cavity, with at least three types film forming agents other than pullulan, including maltodextrins, hydrocolloids and fillers.

Despite the existence of rapidly dissolving orally consumable films in the prior art, there remains room for improvement in such films, and in processes for making them, in particular, such films for the delivery of nitroglycerin.

Nitroglycerin, as referred to herein, is also known as 1,2,3-Propanetriol trinitrate, glyceryl trinitrate, glycerol nitric acid triester, nitroglycerol, trinitroglycerol, glonoine, trinitrin, blasting gelatin, blasting oil, and S.N.G., and is known by numerous commercial brand names, including, but not limited to, Adesitrin, Angibid, Angiolingual, Anginine, Angorin, Aquo-Trinitrosan, Cardamist, Coro-Nitro, Corditrine, Deponit, Diafusor, Gilucor “nitro”, GTN, Klavikordal, Lenitral, Lentonitrina, Millithrol, Minitran, Myoglycerin, Niong, Nitradisc, Nitran, Nitriderm, Nitro-Bid, Nitrobon, Nitrocap, Nitrocap TD, Nitrocine, Nitrocontin, Nitroderm TTS, Nitrodisc, Nitro-Dur, Nitrofortin, Nitro-Gesanit, Nitroglin, Nitroglyn, Nitroguard, Nitrol, Nitrolan, Nitrolande, Nitrolar, Nitro-lent, Nitrolin, Nitrolingual, Nitro Mack, Nitromel, Nitromin, Nitron, Nitronal, Nitronet, Nitrong, Nitro-Pflaster-ratiopharm, NitroPRN, Nitroquick, Nitrorectal, Nitroretard, Nitrosigma, Nitrospan, Nitrostat, Nitrotab, Nitro-Time, Nitrozell retard, Notrong, Nysconitrine, organic nitrate, organic nitrite, Percutol, Perlinganit, Perglottal, Reminitrol, Suscard, Sustac, Sustonit, Transderm-Nitro, Transiderm-Nitro, Tridil, Trinalgon, Trinitrosan and Vasoglyn.

Nitroglycerin is commercially available from a wide variety of sources specifically for pharmaceutical use, including, but not limited to, 3M Pharmaceuticals, Abbott Labs, Aventis Pharmaceuticals, Baxter Healthcare, Cellegy Pharmaceuticals, Inc., DuPont-Merck Pharmaceutical Co., F. Hoffman La-Roche, Ltd., Forest Laboratories, Inc., GlaxoSmithKline, Hoechst Marion Roussel, Kenwood Laboratories, Key Pharmaceuticals, Medley Pharmaceuticals, Merck & Co, Inc., Novartis Pharma AG, Parke-Davis, Pfizer, G. Pohl-Boskamp GmbH & Co., Rhone-Poulene Rorer Pharmaceutical, Inc., Schwartz Pharma AG, Solvay Pharma, Vortech Pharmaceuticals and Warner Lambert Company.

Pure nitroglycerin is a violent explosive which must be handled with great care. The stable form of nitroglycerin crystals melts in the temperate region of 55.4° F. (13° C.). and is extremely unstable as it thaws; liquid nitroglycerin will detonate if subjected to intense heat or percussion. Therefore, nitroglycerin is most useful when its explosive properties are controlled, often by dispersing the compound in an inert substance. Commercially available nitroglycerin may be diluted to a concentration of about 90% by weight, about 80% by weight, about 70% by weight, about 60% by weight, about 50% by weight, about 40% by weight, about 30% by weight, about 20% by weight, about 10% by weight, about 9% by weight, about 8% by weight, about 7% by weight, about 6% by weight, about 5% by weight, about 4% by weight, about 3% by weight, about 2% by weight, about 1% by weight, or less than about 1% by weight, prior to manufacturing into an edible film of the present invention. In one embodiment, nitroglycerin may be diluted to a concentration below 2% by weight prior to use in the methods of the present invention for making edible films. Additionally, in the present invention, it is recommended that certain protective apparel such as gowns, respirators, gloves and goggles, should be worn when working with nitroglycerin to avoid its toxic effects. The skin and mucus membranes readily absorb nitroglycerin and direct skin contact must therefore be avoided. Rapid absorption through the skin makes nitroglycerin a useful drug for the treatment of angina pectoris, but may be harmful to the healthy individual experiencing no oxygen deficiency in the myocardium.

Nitroglycerin may be prepared in aqueous form and is described in U.S. Pat. No. 4,879,308, the entire disclosure of which is incorporated by reference herein, and may also be prepared in non-polar liquid form as described in U.S. Pat. No. 5,869,082, the entire disclosure of which is incorporated by reference herein.

Composition of Films

An embodiment of the invention is a fast dissolving film that comprises a physiologically acceptable amount of nitroglycerin. The expression “physiologically acceptable” amounts of nitroglycerin, as used herein, is intended to encompass an amount or dose, which upon administration to a patient, is adequately tolerated and effective for treatment without causing undue negative side effects, and are physiologically acceptable and compatible with oral films. The amount of nitroglycerin that can be used in the rapidly dissolving films, according to the present invention, is dependent upon the dose needed to provide an effective amount of nitroglycerin. As described herein for nitroglycerin, physiologically acceptable amounts of any other therapeutic agent to be formulated. in the films of the present invention may be determined in a similar manner.

The dosage needed to provide an effective amount of nitroglycerin or any other therapeutic agent may be readily determined by one of ordinary skill in the art using well known techniques, and is typically an amount that will cause an amelioration of symptoms or disease. Specific doses may be adjusted depending on conditions of the disease, the age, body weight, general health, sex, diet of the subject, dose. intervals, excretion rate and combinations with other drugs. As used herein, a therapeutically effective amount of nitroglycerin is an amount in the range of about 0.001 mg to about 1000 mg, or in the range of about 0.01 mg to about 100 mg, or in the range of about 0.05 mg to about 50 mg, or in the range of about 0.1 mg to about 40 mg.

Preparation of Films

The nitroglycerin comprising film of the present invention, or the films of the present invention comprising any other therapeutic agent, in one embodiment comprises at least one film-forming agent and may further comprise water, additional film-forming agents, triglycerides, preservatives, polyethylene oxide compounds, propylene glycol, potentiating agents, saliva stimulating agents, plasticizing agents, cooling agents, surfactants, nitroglycerin stabilizing agents, film stabilizing agents, emulsifying agents, thickening agents, binding agents, buffers, releasing agents, permeation enhancers, sweeteners, additional natural and artificial flavoring agents, coloring agents, coating agents, additional pharmaceutically active agents, antibacterial agents, antiviral agents, other therapeutic agents, and the like.

The film-forming agent used in the films according to the present invention can be any suitable film-forming agent including, but not limited to, pullulan, hydrocolloids, β-glucan, maltodextrin, celluloses, including hydroxypropylmethyl cellulose, hydroxyethyl cellulose, hydroxypropyl cellulose, carboxymethyl cellulose, methylcellulose, hydroxymethylcellulose, hydroxyethylcellulose, polyvinyl pyrrolidone, polyvinyl alcohol, sodium alginate, polyethylene glycol, ethyl cellulose, hydroxypropyl ethyl cellulose, cellulose acetate phthalate, hydroxypropyl methyl cellulose phthalate, natural gums, such as locust bean gum, carrageenan gum, xanthan gum, tragacanth gum, guar gum, acacia gum, arabic gum, karaya, ghatti, tamarind gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein, and mixtures thereof. The film-forming agent used in the films may also include biodegradable polymers, copolymers, block polymers, including, but not limited to, poly(glycolic acid) (PGA), poly(lactic acid) (PLA), polydioxanoes, polyoxalates, poly(.alpha.-esters), polyanhydrides, polyacetates, polycaprolactones, poly(orthoesters), polyamino acids, polyaminocarbonates, polyurethanes, polycarbonates, polyamides, poly(alkyl cyanoacrylates), stereopolymers of L- and D-lactic acid, copolymers of bis(p-carboxyphenoxy) propane acid and sebacic acid, sebacic acid copolymers, copolymers of caprolactone, poly(lactic acid)/poly(glycolic acid)/polyethyleneglycol copolymers, copolymers of polyurethane and poly(lactic acid), copolymers of polyurethane and poly(lactic acid), copolymers of .alpha.-amino acids, copolymers of .alpha.-amino acids and caproic acid, copolymers of .alpha.-benzyl glutamate and polyethylene glycol, copolymers of succinate and poly(glycols), polyphosphazene, polyhydroxy-alkanoates, and any combinations thereof.

In one embodiment of the present invention, at least one film former is pullulan, in amounts ranging from about 0.01 to about 99 wt %, about 30 to about 80 wt %, or from about 45 to about 70 wt % of the film, or from about 60 to about 65 wt % of the film.

In yet another embodiment of the present invention, at least one film former is a hydrocolloid material known in the art for its film-forming properties. The hydrocolloid material may be present in a wide range of concentrations, including, but not limited to, amounts ranging from about 50 to about 90 wt %, or at about 50 to about 80 wt %.

In another embodiment of the present invention, at least one film former is a maltodextrin. The maltodextrin may be present in a wide range of concentrations, including, but not limited to, amounts ranging from between about 5 to about 60 wt %, preferably between about 20 to about 40 wt %, and may be present with a hydrocolloid material, in a range of between about 10 to about 50 wt %, or about 30 to about 40 wt % of the film.

In yet another embodiment of the present invention, at least one film former is a purified β-glucan solution. The β-glucan solution may be used in a wide range of concentrations, including, but not limited to a range of about 10 wt % of the film.

The films comprising nitroglycerin, or films comprising any other therapeutic agent, also may include a triglyceride. Examples of triglycerides include, but are not limited to, vegetable oils such as corn oil, sunflower oil, peanut oil, olive oil, canola oil, soybean oil and mixtures thereof. In one embodiment, the triglyceride is olive oil. The triglyceride is added to the film in amounts from about 0.1 wt % to about 12 wt %, or in a range from about 0.5 to about 9 wt %, of the film.

The films comprising nitroglycerin, or films comprising any other therapeutic agent, also may include a preservative. The preservative may be added in amounts from about 0.001 to about 5 wt %, or from about 0.01 to about 1 wt % of the film. In one embodiment, preservatives include sodium benzoate and potassium sorbate.

The films comprising nitroglycerin, or films comprising any other therapeutic agent, may also include a polyethylene oxide compound. The molecular weight of the polyethylene oxide compound may be within a very broad range, including, but not limited to, ranges from about 50,000 to about 6,000,000. In one embodiment, the polyethylene oxide compound is N-10 available from Union Carbide Corporation. The polyethylene oxide compound may be added in amounts from about 0.1 to about 5 wt %, or from about 0.2 to about 4.0 wt % of the film.

The films comprising nitroglycerin, or films comprising any other therapeutic agent, may also include propylene glycol. The propylene glycol may be added in wide range of amounts, including, but not limited to, from about 1 to about 20 wt %, or from about 5 to about 15 wt % of the film.

The films comprising nitroglycerin may also include a nitroglycerin potentiating agent.. Such nitroglycerin potentiating agents include, but are not limited to, menthol, as disclosed in U.S. Pat. No. 6,559,180, the entire content of which is incorporate by reference herein. Potentating agents for any other therapeutic agent formulated in the films of the present invention may be added, depending on the therapeutic agent in the film.

The films comprising nitroglycerin, or films comprising any other therapeutic agent, also may include saliva stimulating agents. Useful saliva stimulating agents include, but are not limited to, those disclosed in U.S. Pat. No. 4,820,506, which is incorporated by reference herein. Saliva stimulating agents include food acids such as citric, lactic, malic, succinic, ascorbic, adipic, fumaric and tartaric acids. Suitable food acids include, but are not limited to, citric, malic and ascorbic acids. The amount of saliva stimulating agents in the film may be used in a wide range of amounts, including, but not limited to, from about 0.01 to about 12 wt %, or about 1 to about 10 wt %, or about 2.5 to about 6 wt %.

Plasticizing agents including, but not limited to, triacetin may be added to the films comprising nitroglycerin, or films comprising any other therapeutic agent, in a wide range of amounts, including, but not limited to amounts ranging from about 0 to about 20 wt %, or about 0 to about 2 wt %. Other suitable plasticizing agents include, but are not limited to, polyols, such as sorbitol, glycerin, polyethylene glycol, propylene glycol, hydrogenated starch hydrolysates, corn syrups, as well as monoacetin, diacetin, maltitol and mannitol.

Cooling agents including, but not limited to, monomenthyl succinate may be added to the films comprising nitroglycerin, or films comprising any other therapeutic agent, in a wide range of amounts, including, but not limited to amounts ranging from about 0.001 to about 2.0 wt %, or about 0.2 to about 0.4 wt %. A monomenthyl succinate containing cooling agent is available from Mane, Inc. Other suitable cooling agents include, but are not limited to, WS3, WS23, Ultracool II and the like.

Surfactants including, but not limited to, mono and diglycerides of fatty acids and polyoxyethylene sorbitol esters, such as, Atmos 300 and Polysorbate 80 may be added to the films comprising nitroglycerin, or films comprising any other therapeutic agent. The surfactant may be added in a wide range of amounts, including, but not limited to, amounts ranging from about 0.5 to about 15 wt %, or about 1 to about 5 wt % of the film. Other suitable surfactants include, but are not limited to, pluronic acid, sodium lauryl sulfate, and the like.

The films comprising nitroglycerin may also include a nitroglycerin stabilizer in the film. The presence of a stabilizer in the film decreases the loss of nitroglycerin in the film and may prolong shelf-life as well. Suitable stabilizers for nitroglycerin are known in the art, and include, but are not limited to, glyceryl monostearate, which is described in U.S. Pat. No. 6,500,456, the entire content of which is incorporated by reference herein. Stabilizing agents for any other therapeutic agent formulated in the films of the present invention may be added, depending on the therapeutic agent in the film.

Film stabilizing agents including, but not limited to, xanthan gum, locust bean gum and carrageenan, in a wide range of amounts including, but not limited to, amounts ranging from about 0 to about 10 wt %, or about 0.1 to about 2 wt %, may be added to the films comprising nitroglycerin. Other suitable stabilizing agents include, but are not limited to, guar gum and the like.

Emulsifying agents including, but not limited to, lecithin, bentonite, veegum, stearates, triethanolamine stearate, ester derivatives of stearates, palmitates, ester derivatives of palmitates, oleates, ester derivatives of oleates, glycerides, ester derivatives of glycerides, sucrose polyesters, polyglycerolesters, animal waxes, vegetable waxes, synthetic waxes, petroleum, quaternary ammonium compounds, acacia, gelatin, and the like may be added to the films comprising nitroglycerin, or films comprising any other therapeutic agent, in a wide range of amounts, including, but not limited to, amounts ranging from about 0 to about 5 wt %, or about 0.01 to about 0.7 wt % of the film.

Thickening agents including, but not limited to, cellulose ethers, such as methylcellulose, carboxyl methylcellulose, and the like may be added to the films comprising nitroglycerin, or films comprising any other therapeutic agent, in a wide range of amounts, including, but not limited to, amounts ranging from about 0 to about 20 wt %, or about 0.01 to about 5 wt %.

Binding agents including, but not limited to, starch may be added to the films comprising nitroglycerin, or films comprising any other therapeutic agent, in a wide range of amounts, including, but not limited to, amounts ranging from about 0 to about 10 wt %, or about 0.01 to about 2 wt % of the film.

Suitable sweeteners may be included in the films comprising nitroglycerin, or films comprising any other therapeutic agent, include those well known in the art, including both natural and artificial sweeteners. Suitable sweeteners include, but are not limited to:
A. water-soluble sweetening agents such as monosaccharides, disaccharides and polysaccharides such as xylose, ribose, glucose (dextrose), mannose, galactose, fructose (levulose), sucrose (sugar), maltose, invert sugar (a mixture of fructose and glucose derived from sucrose), partially hydrolyzed starch, corn syrup solids, dihydrochalcones, monellin, steviosides, and glycyrrhizin;
B. water-soluble artificial sweeteners such as the soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (acesulfame-K), the free acid form of saccharin, and the like;
C. dipeptide based sweeteners, such as L-aspartic acid derived sweeteners, such as L-aspartyl-L-phenylalanine methyl ester (aspartame) and materials described in U.S. Pat. No. 3,492,131, which is incorporated by reference herein, L-alpha-aspartyl-N-(2,2,4,4-tetramethyl-3-thietanyl)-D-alaninamide hydrate, methyl esters of L-aspartyl-L-phenylglycerin and L-aspartyl-L-2,5,dihydrophenyl-glycine, L-aspartyl-2,5-dihydro-L-phenylalanine, L-aspartyl-L-(1-cyclohexyen)-alanine, and the like;
D. water-soluble sweeteners derived from naturally occurring water-soluble sweeteners, such as a chlorinated derivative of ordinary sugar (sucrose), known, for example, under the product description of sucralose; and
E. protein based sweeteners such as thaumatoccous danielli (Fhaumatin I and II).

In general, an effective amount of auxiliary sweetener is utilized to provide the level of sweetness desired for a particular composition, and this amount will vary with the sweetener selected. This amount will normally be 0.01% to about 10% by weight of the composition when using an easily extractable sweetener. The water-soluble sweeteners described in category A above, are usually used in amounts of about 0.01 to about 10 wt %, and preferably in amounts of about 2 to about 5 wt %. Some of the sweeteners in category A (e.g., glycyrrhizin) can be used in amounts set forth for categories B-E below due to the sweeteners known sweetening ability. In contrast, the sweeteners described in categories B-E are generally used in amounts of about 0.01 to about 10 wt %, or about 2 to about 8 wt %, or about 3 to about 6 wt %. These amounts may be used to achieve a desired level of sweetness independent from the flavor level achieved from any optional flavor oils used.

The nitroglycerin, or films comprising any other therapeutic agent, used in the film can be coated to mask the taste of nitroglycerin, or other therapeutic agent, or to prevent the nitroglycerin, or other therapeutic agents, from numbing or otherwise affecting the tongue or other surfaces in the oral cavity. The coatings that can be used are known to those skilled in the art. These include, but are not limited to, polymers such, as Eudragit® E, cellulosics, such as ethylcellulose, and the like. An additional way to mask the taste of nitroglycerin, or other therapeutic agent, may be by using an ion exchange resin such as Amberlite RP-69, available from Rohm and Haas, and Dow XYS-40010.00, available from the Dow Chemical Co.

Additional natural and artificial flavorings may be chosen from synthetic flavor oils and flavoring aromatics, and/or oils, oleo resins and extracts derived from plants, leaves, flowers, fruits and so forth, and combinations thereof. Representative flavor oils include, but are not limited to, spearmint oil, cinnamon oil, peppermint oil, clove oil, bay oil, thyme oil, cedar leaf oil, oil of nutmeg, oil of sage, and oil of bitter almonds. Also useful are artificial, natural or synthetic fruit flavors such as vanilla, chocolate, coffee, cocoa and citrus oil, including lemon, orange, grape, lime and grapefruit and fruit essences including apple, pear, peach, strawberry, raspberry, cherry, plum, pineapple, apricot and so forth. These flavorings can be used individually or in admixture. Commonly used flavors include mints such as peppermint, artificial vanilla, cinnamon derivatives, and various fruit flavors, whether employed individually or in admixture. Flavorings such as aldehydes and esters including cinnamyl acetate, cinnamaldehyde, citral, diethylacetal, dihydrocarvyl acetate, eugenyl formate, p-methylanisole, and so forth may also be used. Generally, any flavoring or food additive, such as those described in Chemicals Used in Food Processing, publication 1274 by the National Academy of Sciences, pages 63-258, may be used. Further examples of aldehyde flavorings include, but are not limited to, acetaldehyde (apple); benzaldehyde (cherry, almond); cinnamic aldehyde (cinnamon); citral, i.e., alpha citral (lemon, lime); neral, i.e. beta citral (lemon, lime); decanal (orange, lemon); ethyl vanillin (vanilla, cream); heliotropine, i.e., piperonal (vanilla, cream); vanillin (vanilla, cream); alpha-amyl cinnamaldehyde (spicy fruity flavors); butyraldehyde (butter, cheese); valeraldehyde (butter, cheese); citronellal (modifies, many types); decanal (citrus fruits); aldehyde is C-8 (citrus fruits); aldehyde C-9 (citrus fruits); aldehyde C-12 (citrus fruits); 2-ethyl butyraldehyde (berry fruits); hexenal, i.e. trans-2 (berry fruits); tolyl aldehyde (cherry, almond); veratraldehyde (vanilla); 2,6-dimethyl-5-heptenal, i.e. melonal (melon); 2-6-dimethyloctanal (green fruit); and 2-dodecenal (citrus, mandarin); cherry; grape; mixtures thereof, and the like.

The amount of flavoring employed in the film comprising nitroglycerin, or films comprising any other therapeutic agent, may be normally a matter of preference subject to such factors as flavor type, individual flavor, and strength desired. Thus, the amount may be varied in order to obtain the result desired in the final product. Such variations are within the capabilities of those skilled in the art without the need for undue experimentation. In general, amounts of about 0.1 to about 30 wt % are useable with amounts of about 2 to about 25 wt % or amounts from about 8 to about 10 wt %.

The films comprising nitroglycerin, or films comprising any other therapeutic agent, of this invention may also contain coloring agents or colorants. The coloring agents may be used in amounts effective to produce the desired color. The coloring agents useful in the present invention, include pigments such as titanium dioxide, which may be incorporated in amounts of up to about 5 wt %, and preferably less than about 1 wt %. Colorants may also include natural food colors and dyes suitable for food, drug and cosmetic applications. These colorants are known as FD&C dyes and lakes. The materials acceptable for the foregoing spectrum of use are preferably water-soluble, and include FD&C Blue No. 2, which is the disodium salt of 5,5-indigotindisulfonic acid. Similarly, the dye known as Green No. 3 comprises a triphenylmethane dye and is the monosodium salt of 4-[4-N-ethyl-p-sulfobenzylamino)diphenyl-methylene]-[1-N-ethyl-N-p-sulfonium benzyl)-2,5-cyclo-hexadienimine]. A full recitation of all FD&C and D&C dyes and their corresponding chemical structures may be found in the Kirk-Othmer Encyclopedia of Chemical Technology, Volume 5, Pages 857-884, which text is incorporated herein by reference.

In order to prepare a desirable nitroglycerin containing, or films comprising any other therapeutic agent containing, dissolvable matrix for formation into a dosage-form, it may be necessary to combine several general types of components. These components include, but are not limited to, the types of components used to prepare typical confections, the nitroglycerin, and other desired chemically active ingredients such as buffering agents, permeation enhancers, additional pharmaceutically active agents, and the like.

The types of components involved may generally fall into the following categories, including, but not limited to:
1) flavorings,
2) sweeteners,
3) flavor enhancers,
4) releasing agents,
5) buffers,
6) one or more therapeutic agents,
7) dissolvable matrix material, and
8) permeation enhancers.

The components may be a releasable or slowly releasable liquid.

As mentioned above, these components may each be provided in a form which facilitates mixing, such as a dry powder. This provides for convenient combination of the ingredients, even if they happen to be insoluble or otherwise chemically incompatible. All or some of the incipients or inactive ingredients may be on the GRAS list (“generally regarded as safe”).

In certain medications, it may also be desirable to add a lubricating agent in order to release the dosage-form from the mold. Such agents may also provide a certain amount of waterproofing. As mentioned above, the rate of dissolution of the dosage-form within the patient's mouth may be controlled chemically, as well as physically, through the extent of compression of the composition. These lubricating or releasing agents may include, but are not limited to, substances such as compritol 888 (glyceryl behenate), calcium stearate, and sodium stearate. These agents may enhance dissolution or they may inhibit dissolution as necessary.

Lubricating agents may also be useful in those embodiments wherein a powder mixture is funneled into a chute during manufacture. Lubricating agents and surfactants may improve product flow and may avoid static electricity charge buildup within the formulation which may cause the ingredients to separate due to electrostatic forces.

It may also be desirable to include buffering agents within the composition. Buffering agents may provide the ability to place the film comprising nitroglycerin, or films comprising any other therapeutic agent, in the mouth in a favorable pH environment for passage across the mucosal tissues of the mouth, pharynx, and esophagus. Buffering agents incorporated within the composition may be used to affect a pH change in the salival environment of the mouth in order to favor the existence of a unionized form of the nitroglycerin or other active ingredient or drug which more readily moves through the mucosal tissues.

In addition, appropriate pH adjustment may aid in producing a more palatable product with nitroglycerin or other drugs which are either severely acidic (and thus sour) or severely basic (and thus bitter). As a result, a buffer system such as citric acid/sodium citrate may be desirable for addition into the dissolvable matrix. A phosphate buffer system may also be used.

A suitable permeation enhancer capable of improving the drug permeability across the mucosal membrane may also be included in the dissolvable composition. Permeation enhancers may be particularly important when nonlipophilic drugs are used, but may be valuable for lipophilic drugs as well. Examples of typical permeation enhancers which may be used within the scope of the present invention, include, but are not limited to bile salts such as sodium cholate, sodium glycocholate, sodium glycodeoxycholate, taurodeoxycholate, sodium deoxycholate, sodium lithocholate chenocholate, chenodeoxycholate, ursocholate, ursodeoxycholate, hydrodeoxycholate, dehydrocholate, glycochenocholate, taurochenocholate, and taurochenodeoxycholate, as well as sodium dodecyl sulfate (“SDS”), dimethyl sulfoxide (“DMSO”), sodium lauryl sulfate, salts and other derivatives of saturated and unsaturated fatty acids, surfactants, bile salt analogs, derivatives of bile salts. Additionally, synthetic permeation enhancers, as described in U.S. Pat. No. 4,746,508, the entire contents of which are incorporated by reference herein, may also be used.

It will be appreciated by those of ordinary skill in the art that filling and bulking agents of the type known in the art may also be used if desired in the films of the present invention, including, but not limited to, lactose or gelatin.

Added to the dissolvable matrix described above will be the appropriate amount of nitroglycerin. As will be discussed in more detail below, nitroglycerin, or films comprising any other therapeutic agent, is easily incorporated into the matrix compositions to produce the edible or consumable films comprising nitroglycerin, or films comprising other therapeutic agents, of the present invention.

Each of the desired components may be mixed to produce the compositions of the present invention. It may be useful, but not required, to use the method of geometric dilution in mixing the various components. Using this method, the two smallest ingredients by weight (as a proportion of the final product) are first mixed together thoroughly.

When complete mixing has been obtained between those two components, the next smallest ingredient or ingredients by weight equal to the weight of the previous ingredients is added and mixed thoroughly with the existing mixture. This procedure is repeated until all of the components are added to the mix and mixed thoroughly with all other components.

Geometric dilution provides for complete and thorough mixing of all of the components. Using the method described above, there may be less chance for incomplete mixing and uneven distribution of components throughout the mix. Other existing methods may result in incomplete mixing because of the insolubility of the products.

Once complete mixing is accomplished, the mixture may be formed into a solid dissolvable matrix composition. In one embodiment, the mixture may be compressed under relatively high forces to provide a coherent dosage. Compressive forces in the range of from approximately 2,000 Newtons to approximately 5,000 Newtons are suitable, however, any force which is sufficient to compress the ingredients into a coherent, integrated mass could be used.

In other embodiments within the scope of the present invention, the desired constituents may be formed into the dosage-form by dehydration, freeze drying (lyophilization), pouring into a mold, spraying onto a suitable holder, vapor deposition, centrifugation or other known techniques in the art.

When producing the edible films comprising nitroglycerin, or any other therapeutic agent, there may be no need to heat the mixture to a molten mass as has been the practice in the past in forming drug-containing confections. As a result, heat degradation of nitroglycerin, or any other therapeutic agent in the film, may be avoided while good mixing and a uniform product may be provided.

In addition to nitroglycerin, it is readily apparent to those of ordinary skill in the art that other pharmaceutically active agents can be added to the edible films comprising nitroglycerin of the present invention. Alternatively, the pharmaceutically active agents may be formulated in the edible films without nitroglycerin. The expression “pharmaceutically active agents” as used herein is intended to encompass agents. other than foods, which promote a structural and/or functional change in and/or on bodies to which they have been administered. These agents are not particularly limited; however, they should be physiologically acceptable and compatible with the film. Suitable pharmaceutically active agents include, but are not limited to:
A. anti-microbial agents, such as triclosan, cetyl pyridium chloride, domiphen bromide, quaternary ammonium salts, zinc compounds, sanguinarine, fluorides, alexidine, octonidine, EDTA, and the like,
B. non-steroidal anti-inflammatory drugs, such as aspirin, acetaminophen, ibuprofen, ketoprofen, diflunisal, fenoprofen calcium, naproxen, tolmetin sodium, indomethacin, and the like,
C. anti-tussives, such as benzonatate, caramniphen edisylate, menthol, dextromethorphan hydrobromide, chlophedianol hydrochloride, and the like,
D. decongestants, such as pseudoephedrine hydrochloride, phenylepherine, phenylpropanolamine, pseudoephedrine sulfate, and the like,
E. anti-histamines, such as brompheniramine maleate, chlorpheniramine maleate, carbinoxarnine maleate, clemastine fumarate, dexchlorpheniramine maleate, diphenhydramine hydrochloride, diphenylpyraline hydrochloride, azatadine meleate, diphenhydramine citrate, doxylamine succinate, promethazine hydrochloride, pyrilamine maleate, tripelennamine citrate, triprolidine hydrochloride, acrivastine, loratadine, brompheniramine, dexbrompheniramine, and the like,
F. expectorants, such as guaifenesin, ipecac, potassium iodide, terpin hydrate, and the like,
G. anti-diarrheals, such a loperamide, and the like,
H. H₂-antagonists, such as famotidine, ranitidine, and the like,
I. proton pump inhibitors, such as omeprazole, lansoprazole, and the like,
J. general nonselective CNS depressants, such as aliphatic alcohols, barbiturates and the like,
K. general nonselective CNS stimulants, such as caffeine, nicotine, strychnine, picrotoxin, pentylenetetrazol and the like,
L. drugs that selectively modify CNS function, such as phenyhydantoin, phenobarbital, primidone, carbamazepine, ethosukimide, methsuximide, phensuximide, trimethadione, diazepam, benzodiazepines, phenacemide, pheneturide, acetazolamide, sulthiame, bromide, and the like,
M. anti-parkinsonism drugs, such as levodopa, amantadine and the like,
N. narcotic-analgesics, such as alfentanil, benzylmorphine, buprenorphine, clonitazene, codeine, desomorphine, dextromoramide, dimethylthiambutene, eptazocine, ethoheptazine, fentanyl, hydrocodone, hydromorphone, hydroxypethidine, isomethadone, ketobemidone, levorphanol, lofentanil, meperidine, methadone hydrochloride, metopon, morphine, nalbuphine, nalorphine, naloxone, naltrexone norlevorphanol, opium, oxycodone, oxymorphone, papaveretum, phenadoxone, promedol, sufentanil, tilidine, and the like,
O. analgesic-antipyretics, such as salycilates, phenylbutazone, indomethacin, phenacetin, arylsulfanyl and heterosulfanyl derivatives (see U.S. Patent Appl. Serial No. 2003/0078236, incorporated herein by reference), and the like,
P. psychopharmacological drugs, such as chlorpromazine, methotrimeprazine, haloperidol, clozapine, reserpine, imipramine, tranylcypromine, phenelzine, lithium and the like,
Q. anti-hypertension and cardiovascular treatment agents, such as ACE inhibitors, calcium channel blockers, peripheral vasodilators, beta adrenergic blockers, alpha/beta adrenergic blockers, diuretics, digitalis, and isosorbide nitrates, including isosorbide dinitrates and isosorbide mononitrates,
R. dermatological agents, such as acitretin, algesteone acetophenide, ammonium salicylate, anthralin, azathioprime, 6-azauridine, azelaic acid, benzoyl peroxide, bergapten(e), chloroxine, chrysarobip, cyclophosphamide, cyclosporin, cyctol, cyproterone, dichloroacetic acid, doxycycline, etretinate, isotretinoin, 3-O-lauroylpyridoxol diacetate, methotrxate, minocycline, motretinide, piroctone, pyrithione, pyrogallol, resorcinol, retinoic acid, salicylic acid, selenium sulfides, tazarotene, tetroquinone, tioxolone, and the like,
S. glucocorticoids and steroids, such as 21-acetoxypregnenolone, alclometasone, algestone, betamethasoine, beclomethasone, budesonide, clobetasol, corticosterone, cortivazol, deflazacort, dedexamethasone, desoximetasone, difluprednate, enoxolone, fluazacort, flumethasone, fluocortin butyl, flurandrenolide, formocortal, halcinonide, halopredone acetate, hydrocortisone, mazipredone, methylprednisolone, methylparamethasone, prednisolone, predinisone, prednival, prednylidene 21-diethylaminoacetate, tixocortol, triamcinolone, and the like,
T. antimalarial and anti-parasitic agents, such as acedapsone, bebeerines, chirate, chloroguanide, chloroquine, cinchona, gentiopicrin, halofantrine, hydroxychloroquine, mefloquine hydrochloride, mepacrine, 3-methylarsacetin, pamaquine, primaquine, pyrimethamine, quiacrine, quinine, quinocide, quinoline, and sodium arsenate, and the like
U. anti-fungal agents, such as acrisorcin, amorolfine, amphotericin B, azaserine, bifonazole, biphenamine, bromosalicylchlornalide, buclosamide, butoconazole, calcium propionate, candicidin, chlordantoin, chlorphenesin, ciclopirox, cloxyquin, dermostatin, diamthazole, dihydrochloride, econazole, enilconazole, exalamide, fenticonazole, filipin, fluconazole, flucytosine, fungichromin, griseofulvin, hachimycin, halethazole, hexetidine, intraconazol, isoconazole, itraconazole, ketoconazole, loflucarban, lucensomycin, mepartricin, miconazole, naftifine, natamycin, neomycin undecylenate, nifuratel, nystatin, oliogomycins, omoconazole, oxiconazole, pecilocin, potassium iodide, perimycin, salicylanilide, sicanin, sulconazole, terbinafine, terconazole, tioconazole, tubercidin, tolciclate, ujothion, viridin, zinc propionate, and the like,
V. anti-periodontitis agents, such as cevimeline hydrochloride, chlorhexidine, doxycycline, fluoride, minocycline, pilocarpine, tetracycline, triclosan and the like,
W. emetic agents, such as apocodeine, apomorphine, cephaeline, ipecac, sodium chloride, zinc acetate, and the like,
X. treatments for gout, such as allopurinol, carprofen, colchicine, probenecid, sulfinpyrazone, and the like,
Y. treatments for glaucoma, such as acetazolamide, befunolol, betaxolol, burpranolol, carteolol, dapiprazole, dichlorphenamide, dipivefrin, epinephrine, levobunolol, methazolamide, metipranolol, pilocarpine, pindolol, timolol, and the like,
Z. treatments for attention-deficit hyperactivity disorder, such as methylphenidate (Ritalin), dextroamphetamine, pemoline, athomexetine, and the like,
AA. pre-treatment and treatment for exposure to chemical weapons (e.g. nerve agents), such as atropine, pralidoxime (2-PAM), pralidoxime chloride, diazepam, pyridostigmine and the like,
BB. treatments for acute radiation exposure, such as potassium iodide, Prussian Blue and the like,
CC. hemostatic agents, such as adrenalone, adrenochrome, algin, alginic acid, aminochromes, batroxobin, carbazochrome salicylate, cephalins, cotarnine, ellagic acid, ethamsylate, factor viii, factor ix, factor xiii, 1,2-naphthylamine-4-sulfonic acid, oxamarin, oxidized cellulose, styptic collodion, sulmarin, thrombin, thromboplastin, tolonium chloride, tranexamic acid, vasopressin, vitamin k₂, and the like,
DD. treatments for Sjörgren's Syndrome, or dry mouth syndrome, such as pilocarpine (Salagon) and cevimeline hydrochloride (Evoxac), and;
EE. smoking cessation agents, such as nicotine, bupropion HCL, lobeline, clonidine, nortyptaline, and the like.

The nitroglycerin, or other therapeutic agent, in the edible or consumable films of the present invention is prepared to provide a particular dosage per portion of the film. The thickness width and length of the film may be used to calculate the dose contained in the film if the nitroglycerin is uniformly distributed throughout at a known or predetermined concentration. Alternatively, the amount of nitroglycerin, or other therapeutic agent, added to the film ingredients may be adjusted to provide a desired dose when the thickness width and length of the film are uniform.

Other objectives, features, and advantages of the present invention will become apparent from the following specific examples. The examples, while indicating specific embodiments of the invention, are provided by way of illustration only. Accordingly, the present invention also includes those various changes and modifications within the spirit and scope of the invention that may become apparent to those skilled in the art from this detailed description.

### EXAMPLES

The invention will be illustrated in more detail with reference to the following Examples, but it should be understood that the present invention is not deemed to be limited thereto.

### Example 1

The following method is used to prepare films of nitroglycerin, as well as films that comprise other therapeutic agents with or without nitroglycerin:
1. The film-forming ingredients (e.g., xanthan gum, locust bean gum, carrageenan and pullulan) other than Polysorbate 80 and Atmos 300 are mixed and hydrated in hot purified water to form a gel and stored in a refrigerator overnight at a temperature of approximately 4° C. to form preparation A.
2. The coloring agent(s), copper gluconate and sweetener are added to and dissolved in purified water to form preparation B.
3. Preparation B is added to preparation A and mixed well to form preparation C.
4. The flavoring agent(s) is mixed to form preparation D.
5. The polysorbate 80 and Atmos 300 are added to preparation D and mixed well to form preparation E.
6. Preparation E is added to preparation C and mixed well to form preparation F.

Nitroglycerin, or the other therapeutic agent, is added to any of the above-described preparations in the desired amount to yield the desired dosage in the finished film. Preparation F is poured on a mold and cast to form a film of a desired thickness at room temperature. The film is dried under warm air and cut to a desired dimension, packaged and stored.

### Example 2

Edible films comprising nitroglycerin, as well as films that comprise other therapeutic agents with or without nitroglycerin are prepared using a method which comprises the following steps:
1. dissolve copper gluconate, acesulfame K, aspartame, glycerin, sorbitol and dye in purified water to form an aqueous mixture;
2. mix pullulan, xanthan gum, locust bean gum and carrageenan together in powder form to form a powder mixture;
3. add the powder mixture from step B to the aqueous mixture from step A to form a hydrated polymer gel;
4. stir the hydrated polymer from step C at slow speed (about 50-100 RPM) overnight at room temperature;
5. cast the uniform mixture from step D on a suitable backing; and
6. dry the cast mixture to form a film.

Nitroglycerin, or other therapeutic agents, may be added to the mixture at any of Steps A through D at a desired amount to provide a desired dose in the finished film. The finished film is cut to the desired dimensions and stored.

It can be seen, therefore, that the present invention provides a great deal of flexibility in the construction of an appropriate drug-containing edible film. The quantity of drug contained in any edible film can be varied within wide ranges.

### Example 3

Edible films comprising nitroglycerin, or films comprising any other therapeutic agent, may be prepared as follows:
1. Add sodium benzoate and sweeteners to water.
2. Mix locust bean gum, xanthan gum and carrageenan together.
3. Add the gum mixture to the mixture of step 1 and mix until dissolved.
4. Mix nitroglycerin, or the other therapeutic agents, with either water or propylene glycol in an amount to provide the desired dose in the finished film.
5. Add the remaining desired ingredients to the mixture of step 4 or mix the remaining desired ingredients in a separate mixture.
6. Add the mixtures of step 4 and step 5 to the mixture of step 3. Cast and dry to make a film and cut to a size to achieve the desired nitroglycerin dose, or the desired dose of the other therapeutic agents.

### Example 4

Edible films comprising nitroglycerin, or films comprising any other therapeutic agent, may be prepared as follows:
1. Add sodium benzoate to water heated to 50 C. Mix to dissolve.
2. Separately, add Peg 1450, titanium dioxide and nitroglycerin, or other therapeutic agent, to the mixture of step 1, mixing with each addition. The amount of nitroglycerin, or other therapeutic agent, added is the amount that yields the desired dose in the finished film.
3. Mix the locust bean gum, xanthan gum and carrageenan together.
4. Add the gums to the mixture of step 2 and mix until dissolve.
5. Add the remaining ingredients together with heat if needed.
6. Add the mixture of steps 4 and 5 together. Cast and dry to make a film and cut to a size to achieve the desired dose.

The nitroglycerin, or other therapeutic agent, in the edible films of the present invention is prepared to provide a particular dosage per portion of the film. The thickness, width, and length of the film can be used to calculate the dose contained in the film if the nitroglycerin, or other therapeutic agent, is uniformly distributed throughout at a known or predetermined concentration. Alternatively, the amount of nitroglycerin, or other therapeutic agent, added to the film ingredients may be adjusted to provide a desired dose when the thickness width and length of the film are uniform.

### Example 5

Edible films comprising nitroglycerin, or any other therapeutic agent, may be prepared as follows:
1. Add hydrocolloid starch solution to de-ionized water with high shear mixing until clear water is formed.
2. Heat de-ionized water to 40° C. and add protein solution (e.g. gelatin) with slow agitation until protein is dissolved; reducing heat to 30° C.
3. Add mixture of step 1 and step 2 with Sorbo Sorbitol solution and Polysorbate 80 and mix until dissolved.
4. Mix nitroglycerin, or other therapeutic agent, with either water or propylene glycol in an amount to provide the desired dose in the finished film.
5. Add the remaining desired ingredients to the mixture of step 4 or mix the remaining desired ingredients in a separate mixture.
6. Add the mixtures of step 4 and step 5 to the mixture of step 3. Cast onto a polyethylene coated differential release paper using a knife-over-roll coating head, and dry in drying tunnel to make a film and cut to a size to achieve the desired dose.

### Example 6

Edible films comprising nitroglycerin, or any other therapeutic agent, may be prepared as follows:
1. Mix maltodextrin, sodium alginate and 10 microcrystalline cellulose to water heated to boiling while stirring.
2. Cool mixture to a temperature between 35° C. to about 40° C., adding flavor/emulsifier blends, sweeteners, softeners and color to mixture.
3. Mix nitroglycerin, or the other therapeutic agents, with either water or propylene glycol in an amount to provide the desired dose in the finished film.
4. Add the remaining desired ingredients to the mixture of step 3 or mix the remaining desired ingredients in a separate mixture.
5. Add the mixtures of step 3 and step 4 to the mixture of step 2.
6. Spread onto a glass plate by utilizing a draw down blade, and dry solution in an oven for about 15 minutes at 40° C. to make a film and cut to a size to achieve the desired dose.

### Example 7

Edible films comprising nitroglycerin, or any other therapeutic agent, may be prepared as follows:
1. Mix a purified β-glucan in heated water to form a β-glucan solution.
2. Mix nitroglycerin, or the other therapeutic agents, with either water or propylene glycol in an amount to provide the desired dose in the finished film.
3. Add the remaining desired ingredients to the mixture of step 2 or mix the remaining desired ingredients in a separate mixture.
4. Pour liquid mixture onto a heated bomb at 150° C. for 15 minutes to evaporate water from solution.
5. Peel film off hot surface and dry further in an oven at 70° C., and cut to a size to achieve the desired nitroglycerin, or other therapeutic agent, dose.

What is claimed is:

1. A consumable film adapted to dissolve in a mouth of a patient, wherein said film comprises nitroglycerin and a water soluble polymer.

2. The consumable film according to claim 1, wherein said water soluble polymer is selected from the group consisting of pullulan, hydrocolloids, β-glucan, maltodextrin, celluloses, including hydroxypropylmethyl cellulose, hydroxyethyl cellulose, hydroxypropyl cellulose, carboxymethyl cellulose, methylcellulose, hydroxymethylcellulose, hydroxyethylcellulose, polyvinyl pyrrolidone, polyvinyl alcohol, sodium alginate, polyethylene glycol, natural gums, such as locust bean gum, carageenen gum, xanthan gum, tragacanth gum, guar gum, acacia gum, arabic gum, karaya, ghatti, tamarind gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein, and mixtures thereof.

3. The consumable film according to claim 2, wherein said water soluble polymer is pullulan.

4. The consumable film according to claim 3, comprising: about 40 to about 80 wt % pullulan; about 0.01 to about 4 wt % thymol; about 0.01 to about 4 wt % methyl salicylate; about 0.01 to about 4 wt % eucalyptol; and about 0.01 to about 15 wt % menthol.

5. The consumable film according to claim 2, further comprising: about 0.01 to about 5 wt % of at least one stabilizing agent; about 0.001 to about 0.1 wt % of at least one of at least one coloring agent; about 0.1 to about 8 wt % of water; about 0.1 to about 15 wt % of at least one sweetening agent; about 0.1 to about 15 wt % of at least one flavoring agent; about 0.1 to about 4 wt % of at least one cooling agent; and about 0.1 to about 5 wt % of at least one surfactant.

6. The consumable film according to claim 5, wherein said at least one stabilizing agent is selected from the group consisting of xanthan gum, locust bean gum and carrageenan, and said at least one sweetening agent is selected from the group consisting of saccharin, aspartame and acesulfame K.

7. The consumable film according to claim 1, wherein said film does not substantially adhere to itself.

8. The consumable film according to claim 1, further comprising water in an amount from about 3 to about 8 wt %.

9. A method for preparing an edible film comprising nitroglycerin, said method comprising: mixing at least one water soluble film former to provide a film-forming mixture; adding nitroglycerin to the film-forming mixture; casting the film-forming mixture comprising nitroglycerin on a substrate; and drying the cast film to provide said edible film comprising nitroglycerin.

10. The method according to claim 9, wherein at least one surfactant is mixed into said film forming mixture.

11. The method according to claim 9, wherein said drying is conducted until said film has a moisture content of about 3 to about 8 wt %.

12. The method according to claim 9, wherein said film-forming mixture is a powder, which is directly combined with an aqueous solution comprising nitroglycerin to form a hydrated polymer gel.

13. The method according to claim 12, wherein said hydrated polymer gel is formed without heating.

14. The method according to claim 13, wherein said hydrated polymer gel is stirred at room temperature for about 2 to about 48 hours.

15. A non-self-adhering film comprising nitroglycerin produced according to the method of claim 9.

16. The method according to claim 9, wherein the water soluble film former is selected from the group consisting of pullulan, hydrocolloids, β-glucan, maltodextrin, celluloses, including hydroxypropylmethyl cellulose, hydroxyethyl cellulose, hydroxypropyl cellulose, carboxymethyl cellulose, methylcellulose, hydroxymethylcellulose, hydroxyethylcellulose, polyvinyl pyrrolidone, polyvinyl alcohol, sodium alginate, polyethylene glycol, natural gums, such as locust bean gum, carageenen gum, xanthan gum, tragacanth gum, guar gum, acacia gum, arabic gum, karaya, ghatti, tamarind gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein, and mixtures thereof.

17. The method according to claim 16, wherein said water soluble film former is pullulan.

18. A consumable film comprising nitroglycerin adapted to dissolve in the mouth of a patient, wherein said film comprises nitroglycerin in a single layer including pullulan and at least one additional pharmaceutical agent.

19. The consumable film according to claim 18, wherein said pharmaceutical agent is selected from the group consisting of anti-microbial agents, non-steroidal anti-inflammatory drugs, anti-tussives, decongestants, anti-histamines, expectorants, anti-diarrheals, H2-antagonists, proton pump inhibitors, general nonselective CNS depressants, general nonselective CNS stimulants, drugs that selectively modify CNS function, anti-parkinsonism drugs, narcotic-analgesics, analgesic-antipyretics, psychopharmacological drugs, anti-hypertension and cardiovascular treatment agents, dermatological agents, glucocorticoids and steroids, antimalarial and anti-parasitic agents, anti-fingal agents, anti-periodontitis agents, emetic agents, treatments for gout, treatments for glaucoma, treatments for attention-deficit hyperactivity disorder, pre-treatment and treatment for exposure to chemical weapons, treatments for acute radiation exposure, hemostatic agents, and treatments for Sjörgren's Syndrome, smoking cessation agents, and mixtures thereof.

20. The consumable film according to claim 19, wherein the anti-microbial agent is selected from the group consisting of triclosan, cetyl pyridium chloride, domiphen bromide, quaternary ammonium salts, zinc compounds, sanguinarine, fluorides, alexidine, octonidine, EDTA and mixtures thereof.

21. The consumable film according to claim 19, wherein the non-steroidal anti-inflammatory drug is selected from the group consisting of aspirin, acetaminophen, ibuprofen, diflunisal, fenoprofen calcium, naproxen, tolmetin sodium, indomethacin, and mixtures thereof.

22. The consumable film according to claim 19, wherein the anti-tussive is selected from the group consisting of benzonatate, caramiphen edisylate, dextromethorphan hydrobromide, chlophedianol hydrochloride and mixtures thereof.

23. The consumable film according to claim 19, wherein the decongestant, is selected from the group consisting of pseudoephedrine hydrochloride, phenylepherine, phenylpropanolamine and mixtures thereof.

24. The consumable film according to claim 19, wherein the anti-histamine is selected from the group consisting of brompheniramine maleate, chlorpheniramine maleate, carbinoxamine maleate, clemastine fumarate, dexchlorpheniramine maleate, diphenhydramine hydrochloride, diphenhydramine citrate, diphenylpyraline hydrochloride, doxylamine succinate, promethazine hydrochloride, pyrilamine maleate, tripelennamine citrate, triprolidine hydrochloride and mixtures thereof.

25. The consumable film according to claim 19, wherein the expectorant is selected from the group consisting of guaifenesin, ipecac, potassium iodide, terpin hydrate and mixtures thereof.

26. The consumable film according to claim 19, wherein the anti-diarrheal is loperamide.

27. The consumable film according to claim 19, wherein the H2-antagonist is selected from the group consisting of famotidine, ranitidine and mixtures thereof.

28. The consumable film according to claim 19, wherein the proton pump inhibitor is selected from the group consisting of omeprazole, lansoprazole, and mixtures thereof.

29. The consumable film according to claim 19, wherein the general nonselective CNS depressant is selected from the group consisting of aliphatic alcohols, barbiturates and mixtures thereof.

30. The consumable film according to claim 19, wherein the general nonselective CNS stimulant is selected from the group consisting of caffeine, nicotine, strychnine, picrotoxin, pentylenetetrazol and mixtures thereof.

31. The consumable film according to claim 19, wherein the drug that selectively modifies CNS function is selected from the group consisting of phenyhydantoin, phenobarbital, primidone, carbamazepine, ethosukimide, methsuximide, phensuximide, trimethadione, diazepam, benzodiazepines, phenacemide, pheneturide, acetazolamide, sulthiame, bromide, and mixtures thereof.

32. The consumable film according to claim 19, wherein the anti-parkinsonism drug is selected from the group consisting of levodopa, amantadine and mixtures thereof.

33. The consumable film according to claim 19, wherein the narcotic-analgesic is selected from the group consisting of alfentanil, benzylmorphine, buprenorphine, clonitazene, codeine, desomorphine, dextromoramide, dimethylthiambutene, eptazocine, ethoheptazine, fentanyl, hydrocodone, hydromorphone, hydroxypethidine, isomethadone, ketobemidone, levorphanol, lofentanil, meperidine, methadone hydrochloride, metopon, morphine, nalbuphine, nalorphine, naloxone, naltrexone norlevorphanol, opium, oxycodone, oxymorphone, papaveretum, phenadoxone, promedol, sufentanil, tilidine, and mixtures thereof.

34. The consumable film according to claim 19, wherein the analgesic-antipyretic is selected from the group consisting of salycilates, phenylbutazone, indomethacin, phenacetin, arylsulfanyl derivatives, heteroarylsulfanyl derivatives, and mixtures thereof.

35. The consumable film according to claim 19, wherein the psychopharmacological drug is selected from the group consisting of chlorpromazine, methotrimeprazine, haloperidol, clozapine, reserpine, imipramine, tranylcypromine, phenelzine, lithium and mixtures thereof.

36. The consumable film according to claim 19, wherein the anti-hypertension and cardiovascular treatment agent is selected from the group consisting of ACE inhibitors, calcium channel blockers, peripheral vasodilators, beta adrenergic blockers, alphalbeta adrenergic blockers, diuretics, digitalis, and isosorbide nitrates, including isosorbide dinitrates and isosorbide mononitrates, and mixtures thereof.

37. The consumable film according to claim 19, wherein the dermatological agent is selected from the group consisting of acitretin, algesteone acetophenide, ammonium salicylate, anthralin, azathioprime, 6-azauridine, azelaic acid, benzoyl peroxide, bergapten(e), chloroxine, chrysarobin, cyclophosphamide, cyclosporin, cyctol, cyproterone, dichloroacetic acid, doxycycline, etretinate, isotretinoin, 3-O-lauroylpyridoxol diacetate, methotrxate, minocycline, motretinide, piroctone, pyrithione, pyrogallol, resorcinol, retinoic acid, salicylic acid, selenium sulfides, tazarotene, tetroquinone, tioxolone, and mixtures thereof.

38. The consumable film according to claim 19, wherein the glucocorticoid and steroid is selected from the group consisting of 21-acetoxypregnenolone, alclometasone, algestone, betamethasoine, beclomethasone, budesonide, clobetasol, corticosterone, cortivazol, deflazacort, dedexamethasone, desoximetasone, difluprednate, enoxolone, fluazacort, flumethasone, fluocortin butyl, flurandrenolide, formocortal, halcinonide, halopredone acetate, hydrocortisone, mazipredone, methylprednisolone, methylparamethasone, prednisolone, predinisone, prednival, prednylidene 21-diethylaminoacetate, tixocortol, triamcinolone, and mixtures thereof.

39. The consumable film according to claim 19, wherein the antimalarial and anti-parasitic agent is selected from the group consisting of acedapsone, bebeerines, chirate, chloroguanide, chloroquine, cinchona, gentiopicrin, halofantrine, hydroxychloroquine, mefloquine hydrochloride, mepacrine, 3-methylarsacetin, pamaquine, primaquine, pyrimethamine, quiacrine, quinine, quinocide, quinoline, and sodium arsenate, and mixtures thereof.

40. The consumable film according to claim 19, wherein the anti-fungal agent is selected from the group consisting of acrisorcin, amorolfine, amphotericin B, azaserine, bifonazole, biphenamine, bromosalicylchlornalide, buclosamide, butoconazole, calcium propionate, candicidin, chlordantoin, chlorphenesin, ciclopirox, cloxyquin, dermostatin, diamthazole, dihydrochloride, econazole, enilconazole, exalamide, fenticonazole, filipin, fluconazole, flucytosine, fungichromin, griseofulvin, hachimycin, halethazole, hexetidine, intraconazol, isoconazole, itraconazole, ketoconazole, loflucarban, lucensomycin, mepartricin, miconazole, naftifine, natamycin, neomycin undecylenate, nifuratel, nystatin, oliogomycins, omoconazole, oxiconazole, pecilocin, potassium iodide, perimycin, salicylanilide, sicanin, sulconazole, terbinafme, terconazole, tioconazole, tubercidin, tolciclate, ujothion, viridin, zinc propionate, and mixtures thereof.

41. The consumable film according to claim 19, wherein the anti-periodontitis agent is selected from the group consisting of cevimeline hydrochloride, chlorhexidine, doxycycline, fluoride, minocycline, pilocarpine, tetracycline, triclosan and mixtures thereof.

42. The consumable film according to claim 19, wherein the emetic agent is selected from the group consisting of apocodeine, apomorphine, cephaeline, ipecac, sodium chloride, zinc acetate, and mixtures thereof.

43. The consumable film according to claim 19, wherein the treatment for gout is selected from the group consisting of allopurinol, carprofen, colchicine, probenecid, sulfinpyrazone, and mixtures thereof.

44. The consumable film according to claim 19, wherein the treatment for glaucoma is selected from the group consisting of acetazolamide, befunolol, betaxolol, burpranolol, carteolol, dapiprazole, dichlorphenamide, dipivefrin, epinephrine, levobunolol, methazolamide, metipranolol, pilocarpine, pindolol, timolol, and mixtures thereof.

45. The consumable film according to claim 19, wherein the treatment for attention-deficit hyperactivity disorder is selected from the group consisting of methylphenidate (Ritalin), dextroamphetamine, pemoline, athomexetine, and mixtures thereof.

46. The consumable film according to claim 19, wherein the pre-treatment and treatment for exposure to chemical weapons is selected from the group consisting of atropine, pralidoxime (2-PAM), pralidoxime chloride, diazepam, pyridostigmine and mixtures thereof.

47. The consumable film according to claim 19, wherein the treatment for acute radiation exposure is selected from the group consisting of potassium iodide, Prussian Blue and mixtures thereof.

48. The consumable film according to claim 19, wherein the hemostatic agent is selected from the group consisting of adrenalone, adrenochrome, algin, alginic acid, aminochromes, batroxobin, carbazochrome salicylate, cephalins, cotarnine, ellagic acid, ethamsylate, factor viii, factor ix, factor xiii, 1,2-naphthylamine-4-sulfonic acid, oxamarin, oxidized cellulose, styptic collodion, sulmarin, thrombin, thromboplastin, tolonium chloride, tranexamic acid, vasopressin, vitamin k2 and mixtures thereof.

49. The consumable film according to claim 19, wherein the treatment for Sjorgren's Syndrome is selected from the group consisting of pilocarpine (Salagon) and cevimeline hydrochloride (Evoxac), and mixtures thereof.

50. The consumable film according to claim 19, wherein the smoking cessation agent is selected from the group consisting of nicotine, bupropion HCL, lobeline, clonidine, and nortyptaline.

51. A method for delivering an effective amount of nitroglycerin to the oral cavity comprising introducing in the oral cavity a rapidly dissolving edible film comprising pullulan and nitroglycerin.

52. The method according to claim 51, wherein the amount of pullulan in the film is from about 40 to about 80 wt %.

53. The method according to claim 51, wherein the amount of nitroglycerin in the film is from about 0.0001 to about 90 wt %.

54. A method for delivering an effective amount of nitroglycerin to the oral cavity comprising introducing in the oral cavity the consumable film according to claim 1.

55. An edible film comprising nitroglycerin for use in transmucosal delivery of nitroglycerin to a patient, said film comprising:
a) a binding agent which is dissolvable in the mouth of the patient; and,
b) a pharmacologically effective dose of nitroglycerin dispersed in the binding agent to form a mixture that is fashioned into a film such that when the film dissolves in the mouth of the patient, the pharmacologically effective dose of nitroglycerin is released.

56. A consumable film adapted to dissolve in a mouth of a patient, wherein said film comprises one or more therapeutic agents selected from a group consisting of anti-microbial agents, non-steroidal anti-inflammatory drugs, anti-inflammatory drugs, anti-tussives, decongestants, anti-histamines, expectorants, anti-diarrheals, H2-antagonists, proton pump inhibitors, general nonselective CNS depressants, general nonselective CNS stimulants, drugs that selectively modify CNS function, anti-parkinsonism drugs, narcotic-analgesics, analgesic-antipyretics, psychopharmacological drugs, anti-hypertension and cardiovascular treatment agents, dermatological agents, glucocorticoids and steroids, antimalarial and anti-parasitic agents, anti-fungal agents, anti-periodontitis agents, emetic agents, treatments for gout, treatments for glaucoma, treatments for attention-deficit hyperactivity disorder, pre-treatment and treatment for exposure to chemical weapons, treatments for acute radiation exposure, hemostatic agents, treatments for Sjörgren's Syndrome and smoking cessation agents and a water soluble polymer.

57. The consumable film according to claim 56, wherein said water soluble polymer is selected from the group consisting of pullulan, hydrocolloids, β-glucan, maltodextrin, celluloses, including hydroxypropylmethyl cellulose, hydroxyethyl cellulose, hydroxypropyl cellulose, carboxymethyl cellulose, methylcellulose, hydroxymethylcellulose, hydroxyethylcellulose, polyvinyl pyrrolidone, polyvinyl alcohol, sodium alginate, polyethylene glycol, natural gums, such as locust bean gum, carageenen gum, xanthan gum, tragacanth gum, guar gum, acacia gum, arabic gum, karaya, ghatti, tamarind gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein, and mixtures thereof.

58. The consumable film according to claim 57, wherein said water soluble polymer is pullulan.

59. The consumable film according to claim 57, comprising: about 40 to about 80 wt % pullulan; about 0.01 to about 4 wt % thymol; about 0.01 to about 4 wt % methyl salicylate; about 0.01 to about 4 wt % eucalyptol; and about 0.01 to about 15 wt % menthol.

60. The consumable film according to claim 57, further comprising: about 0.01 to about 5 wt % of at least one stabilizing agent; about 0.001 to about 0.1 wt % of at least one of at least one coloring agent; about 0.1 to about 8 wt % of water; about 0.1 to about 15 wt % of at least one sweetening agent; about 0.1 to about 15 wt % of at least one flavoring agent; about 0.1 to about 4 wt % of at least one cooling agent; and about 0.1 to about 5 wt % of at least one surfactant.

61. The consumable film according to claim 60, wherein said at least one stabilizing agent is selected from the group consisting of xanthan gum, locust bean gum and carrageenan, and said at least one sweetening agent is selected from the group consisting of saccharin, aspartame and acesulfame K.

62. The consumable film according to claim 56, wherein said film does not substantially adhere to itself.

63. The consumable film according to claim 56, further comprising water in an amount from about 3% to about 8 wt %.

64. A method for preparing an edible film comprising one or more therapeutic agents selected from a group consisting of anti-microbial agents, non-steroidal anti-inflammatory drugs, anti-inflammatory drugs, anti-tussives, decongestants, anti-histamines, expectorants, anti-diarrheals, H2-antagonists, proton pump inhibitors, general nonselective CNS depressants, general nonselective CNS stimulants, drugs that selectively modify CNS function, anti-parkinsonism drugs, narcotic-analgesics, analgesic-antipyretics, psychopharmacological drugs, anti-hypertension and cardiovascular treatment agents, dermatological agents, glucocorticoids and steroids, antimalarial and anti-parasitic agents, anti-fungal agents, anti-periodontitis agents, emetic agents, treatments for gout, treatments for glaucoma, treatments for attention-deficit hyperactivity disorder, pre-treatment and treatment for exposure to chemical weapons, treatments for acute radiation exposure, hemostatic agents, treatments for Sjörgren's Syndrome and smoking cessation agents, said method comprising: mixing at least one water soluble film former to provide a film-forming mixture; adding the therapeutic agent(s) to the film-forming mixture; casting the film-forming mixture comprising the therapeutic agent(s) on a substrate; and drying the cast film to provide said edible film comprising the therapeutic agent(s).

65. The method according to claim 64, wherein at least one surfactant is mixed into said film forming mixture.

66. The method according to claim 64, wherein said drying is conducted until said film has a moisture content of about 3 to about 8 wt %.

67. The method according to claim 64, wherein said film-forming mixture is a powder, which is directly combined with an aqueous solution comprising one or more therapeutic agents -selected from a group consisting of anti-microbial agents, non-steroidal anti-inflammatory drugs, anti-inflammatory drugs, anti-tussives, decongestants, anti-histamines, expectorants, anti-diarrheals, H2-antagonists, proton pump inhibitors, general nonselective CNS depressants, general nonselective CNS stimulants, drugs that selectively modify CNS function, anti-parkinsonism drugs, narcotic-analgesics, analgesic-antipyretics, psychopharmacological drugs, anti-hypertension and cardiovascular treatment agents, dermatological agents, glucocorticoids and steroids, antimalarial and anti-parasitic agents, anti-fungal agents, anti-periodontitis agents, emetic agents, treatments for gout, treatments for glaucoma, treatments for attention-deficit hyperactivity disorder, pre-treatment and treatment for exposure to chemical weapons, treatments for acute radiation exposure, hemostatic agents, treatments for Sjörgren's Syndrome and smoking cessation agents, to form a hydrated polymer gel.

68. The method according to claim 67, wherein said hydrated polymer gel is formed without heating.

69. The method according to claim 68, wherein said hydrated polymer gel is stirred at room temperature for about 2 to about 48 hours.

70. A non-self-adhering film comprising one or more therapeutic agents selected from a group consisting of anti-microbial agents, non-steroidal anti-inflammatory drugs, anti-inflammatory drugs, anti-tussives, decongestants, anti-histamines, expectorants, anti-diarrheals, H2-antagonists, proton pump inhibitors, general nonselective CNS depressants, general nonselective CNS stimulants, drugs that selectively modify CNS function, anti-parkinsonism drugs, narcotic-analgesics, analgesic-antipyretics, psychopharmacological drugs, anti-hypertension and cardiovascular treatment agents, dermatological agents, glucocorticoids and steroids, antimalarial and anti-parasitic agents, anti-fungal agents, anti-periodontitis agents, emetic agents, treatments for gout, treatments for glaucoma, treatments for attention-deficit hyperactivity disorder, pre-treatment and treatment for exposure to chemical weapons, treatments for acute radiation exposure, hemostatic agents, treatments for Sjörgren's Syndrome and smoking cessation agents, produced according to the method of claim 64.

71. The method according to claim 64, wherein the water soluble film former is selected from the group consisting of pullulan, hydrocolloids, β-glucan, maltodextrin, celluloses, including hydroxypropylmethyl cellulose, hydroxyethyl cellulose, hydroxypropyl cellulose, carboxymethyl cellulose, methylcellulose, hydroxymethylcellulose, hydroxyethylcellulose, polyvinyl pyrrolidone, , polyvinyl alcohol, sodium alginate, polyethylene glycol, natural gums, such as locust bean gum, carageenen gum, xanthan gum, tragacanth gum, guar gum, acacia gum, arabic gum, karaya, ghatti, tamarind gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein, and mixtures thereof.

72. The method according to claim 71, wherein said water soluble film former is pullulan.

73. A consumable film adapted to dissolve in the mouth of a patient, wherein said film comprises one or more therapeutic agents selected from a group consisting of anti-microbial agents, non-steroidal anti-inflammatory drugs, anti-inflammatory drugs, anti-tussives, decongestants, anti-histamines, expectorants, anti-diarrheals, H2-antagonists, proton pump inhibitors, general nonselective CNS depressants, general nonselective CNS stimulants, drugs that selectively modify CNS function, anti-parkinsonism drugs, narcotic-analgesics, analgesic-antipyretics, psychopharmacological drugs, anti-hypertension and cardiovascular treatment agents, dermatological agents, glucocorticoids and steroids, antimalarial and anti-parasitic agents, anti-fungal agents, anti-periodontitis agents, emetic agents, treatments for gout, treatments for glaucoma, treatments for attention-deficit hyperactivity disorder, pre-treatment and treatment for exposure to chemical weapons, treatments for acute radiation exposure, hemostatic agents, treatments for Sjörgren's Syndrome and smoking cessation agents, in a single layer including pullulan.

74. The consumable film according to claim 73, wherein the anti-microbial agent is selected from the group consisting of triclosan, cetyl pyridium chloride, domiphen bromide, quaternary ammonium salts, zinc compounds, sanguinarine, fluorides, alexidine, octonidine, EDTA and mixtures thereof.

75. The consumable film according to claim 73, wherein the non-steroidal anti-inflammatory. drug is selected from the group consisting of aspirin, acetaminophen, ibuprofen, diflunisal, fenoprofen calcium, naproxen, tolmetin sodium, indomethacin, and mixtures thereof.

76. The consumable film according to claim 73, wherein the anti-tussive is selected from the group consisting of benzonatate, caramiphen edisylate, dextromethorphan hydrobromide, chlophedianol hydrochloride and mixtures thereof.

77. The consumable film according to claim 73, wherein the decongestant is selected from the group consisting of pseudoephedrine hydrochloride, phenylepherine, phenylpropanolamine and mixtures thereof.

78. The consumable film according to claim 73, wherein the anti-histamine is selected from the group consisting of brompheniramine maleate, chlorpheniramine maleate, carbinoxamine maleate, clemastine fumarate, dexchlorpheniramine maleate, diphenhydramine hydrochloride, diphenhydramine citrate, diphenylpyraline hydrochloride, doxylamine succinate, promethazine hydrochloride, pyrilamine maleate, tripelennamine citrate, triprolidine hydrochloride and mixtures thereof.

79. The consumable film according to claim 73, wherein the expectorant is selected from the group consisting of guaifenesin, ipecac, potassium iodide, terpin hydrate and mixtures thereof.

80. The consumable film according to claim 73, wherein the anti-diarrheal is loperamide.

81. The consumable film according to claim 73, wherein the H₂-antagonist is selected from the group consisting of famotidine, ranitidine and mixtures thereof.

82. The consumable film according to claim 73, wherein the proton pump inhibitor is selected from the group consisting of omeprazole, lansoprazole, and mixtures thereof.

83. The consumable film according to claim 73, wherein the general nonselective CNS depressant is selected from the group consisting of aliphatic alcohols, barbiturates and mixtures thereof.

84. The consumable film according to claim 73, wherein the general nonselective CNS stimulant is selected from the group consisting of caffeine, nicotine, strychnine, picrotoxin, pentylenetetrazol and mixtures thereof.

85. The consumable film according to claim 73, wherein the drug that selectively modifies CNS function is selected from the group consisting of phenyhydantoin, phenobarbital, primidone, carbamazepine, ethosukimide, methsuximide, phensuximide, trimethadione, diazepam, benzodiazepines, phenacemide, pheneturide, acetazolamide, sulthiame, bromide, and mixtures thereof.

86. The consumable film according to claim 73, wherein the anti-parkinsonism drug is selected from the group consisting of levodopa, amantadine and mixtures thereof.

87. The consumable film according to claim 73, wherein the narcotic-analgesic is selected from the group consisting of alfentanil, benzylmorphine, buprenorphine, clonitazene, codeine, desomorphine, dextromoramide, dimethylthiambutene, eptazocine, ethoheptazine, fentanyl, hydrocodone, hydromorphone, hydroxypethidine, isomethadone, ketobemidone, levorphanol, lofentanil, meperidine, methadone hydrochloride, metopon, morphine, nalbuphine, nalorphine, naloxone, naltrexone norlevorphanol, opium, oxycodone, oxymorphone, papaveretum, phenadoxone, promedol, sufentanil, tilidine, and mixtures thereof.

88. The consumable film according to claim 73, wherein the analgesic-antipyretic is selected from the group consisting of salycilates, phenylbutazone, indomethacin, phenacetin, arylsulfanyl derivatives, heteroarylsulfanyl derivatives, and mixtures thereof.

89. The consumable film according to claim 73, wherein the psychopharmacological drug is selected from the group consisting of chlorpromazine, methotrimeprazine, haloperidol, clozapine, reserpine, imipramine, tranylcypromine, phenelzine, lithium and mixtures thereof.

90. The consumable film according to claim 73, wherein the anti-hypertension and cardiovascular treatment agent is selected from the group consisting of ACE inhibitors, calcium channel blockers, peripheral vasodilators, beta adrenergic blockers, alpha/beta adrenergic blockers, diuretics, digitalis, and isosorbide nitrates, including isosorbide dinitrates and isosorbide mononitrates, and mixtures thereof.

91. The consumable film according to claim 73, wherein the dermatological agent is selected from the group consisting of acitretin, algesteone acetophenide, ammonium salicylate, anthralin, azathioprime, 6-azauridine, azelaic acid, benzoyl peroxide, bergapten(e), chloroxine, chrysarobin, cyclophosphamide, cyclosporin, cyctol, cyproterone, dichloroacetic acid, doxycycline, etretinate, isotretinoin, 3-O-lauroylpyridoxol diacetate, methotrxate, minocycline, motretinide, piroctone, pyrithione, pyrogallol, resorcinol, retinoic acid, salicylic acid, selenium sulfides, tazarotene, tetroquinone, tioxolone, and mixtures thereof.

92. The consumable film according to claim 73, wherein the glucocorticoid and steroid is selected from the group consisting of 21-acetoxypregnenolone, alclometasone, algestone, betamethasoine, beclomethasone, budesonide, clobetasol, corticosterone, cortivazol, deflazacort, dedexamethasone, desoximetasone, difluprednate, enoxolone, fluazacort, flumethasone, fluocortin butyl, flurandrenolide, formocortal, halcinonide, halopredone acetate, hydrocortisone, mazipredone, methylprednisolone, methylparamethasone, prednisolone, predinisone, prednival, prednylidene 21-diethylaminoacetate, tixocortol, triamcinolone, and mixtures thereof.

93. The consumable film according to claim 73, wherein the antimalarial and anti-parasitic agent is selected from the group consisting of acedapsone, bebeerines, chirate, chloroguanide, chloroquine, cinchona, gentiopicrin, halofantrine, hydroxychloroquine, mefloquine hydrochloride, mepacrine, 3-methylarsacetin, pamaquine, primaquine, pyrimethamine, quiacrine, quinine, quinocide, quinoline, and sodium arsenate, and mixtures thereof.

94. The consumable film according to claim 73, wherein the anti-fungal agent is selected from the group consisting of acrisorcin, amorolfine, amphotericin B, azaserine, bifonazole, biphenamine, bromosalicylchlomalide, buclosamide, butoconazole, calcium propionate, candicidin, chlordantoin, chlorphenesin, ciclopirox, cloxyquin, dermostatin, diamthazole, dihydrochloride, econazole, enilconazole, exalamide, fenticonazole, filipin, fluconazole, flucytosine, fungichromin, griseofulvin, hachimycin, halethazole, hexetidine, intraconazol, isoconazole, itraconazole, ketoconazole, loflucarban, lucensomycin, mepartricin, miconazole, naftifine, natamycin, neomycin undecylenate, nifuratel, nystatin, oliogomycins, omoconazole, oxiconazole, pecilocin, potassium iodide, perimycin, salicylanilide, sicanin, sulconazole, terbinafine, terconazole, tioconazole, tubercidin, tolciclate, ujothion, viridin, zinc propionate, and mixtures thereof.

95. The consumable film according to claim 73, wherein the anti-periodontitis agent is selected from the group consisting of cevimeline hydrochloride, chlorhexidine, doxycycline, fluoride, minocycline, pilocarpine, tetracycline, triclosan and mixtures thereof.

96. The consumable film according to claim 73, wherein the emetic agent is selected from the group consisting of apocodeine, apomorphine, cephaeline, ipecac, sodium chloride, zinc acetate, and mixtures thereof.

97. The consumable film according to claim 73, wherein the treatment for gout is selected from the group consisting of allopurinol, carprofen, colchicine, probenecid, sulfinpyrazone, and mixtures thereof.

98. The consumable film according to claim 73, wherein the treatment for glaucoma is selected from the group consisting of acetazolamide, befunolol, betaxolol, burpranolol, carteolol, dapiprazole, dichlorphenamide, dipivefrin, epinephrine, levobunolol, methazolaminde, metipranolol, pilocarpine, pindolol, timolol, and mixtures thereof.

99. The consumable film according to claim 73, wherein the treatment for attention-deficit hyperactivity disorder is selected from the group consisting of methylphenidate (Ritalin), dextroamphetamine, pemoline, athomexetine, and mixtures thereof.

100. The consumable film according to claim 73, wherein the pre-treatment and treatment for exposure to chemical weapons is selected from the group consisting of atropine, pralidoxime (2-PAM), pralidoxime chloride, diazepam, pyridostigrnine and mixtures thereof.

101. The consumable film according to claim 73, wherein the treatment for acute radiation exposure is selected from the group consisting of potassium iodide, Prussian Blue and mixtures thereof.

102. The consumable film according to claim 73, wherein the hemostatic agent is selected from the group consisting of adrenalone, adrenochrome, algin, alginic acid, aminochromes, batroxobin, carbazochrome salicylate, cephalins, cotarnine, ellagic acid, ethamsylate, factor viii, factor ix, factor xiii, 1,2-naphthylamine-4-sulfonic acid, oxamarin, oxidized cellulose, styptic collodion, sulmarin, thrombin, thromboplastin, tolonium chloride, tranexamic acid, vasopressin, vitamin k₂ and mixtures thereof.

103. The consumable film according to claim 73, wherein the treatment for Sjorgren's Syndrome is selected from the group consisting of pilocarpine (Salagon) and cevimeline hydrochloride (Evoxac), and mixtures thereof.

104. The consumable film according to claim 73, wherein the smoking cessation agents is selected from the group consisting of nicotine, bupropion HCL, lobeline, clonidine, and nortyptaline.
